(19) United States
(12) Patent Application Publication
(10) Pub. No.: US 2022/0020462 A1
(43) Pub. Date: Jan. 20, 2022

(54) GLOBAL TELEMEDICINE SYSTEM FOR ISSUING COUNTRY-CUSTOMIZED PRESCRIPTION

(71) Applicant: HICARENET INC., Seoul (KR)

(72) Inventors: Hong Jin KIM, Gyeonggi-do (KR); Gwun Il Park, Seoul (KR); Ji Yeon Baek, Seoul (KR)

(73) Assignee: HICARENET INC., Seoul (KR)

(21) Appl. No.: 17/125,310

(22) Filed: Dec. 17, 2020

(30) Foreign Application Priority Data
Jul. 15, 2020 (KR) 10-2020-0087384

(51) Int. Cl. G16H 20/10; G16H 40/67; G16H 40/20; G16H 70/40; G06Q 50/26; G06Q 30/00; G16H 50/70; G16H 50/20; G06N 20/00; A61B 5/00; G06F 40/42

(52) U.S. Cl. CPC G16H 20/10; G16H 40/67; G16H 40/20; G16H 70/40; G06Q 50/26; G06Q 30/0607; G16H 50/70; G16H 50/20; G06N 20/00; A61B 5/7465; G06F 40/42; G06Q 30/018

(57) ABSTRACT

A prescription with alternative medications purchasable in a residing country of a patient receiving a telemedicine service, based on a prescription issued by a hospital or doctor of a country providing the telemedicine service, is provided to the patient such that the patient receives suitable and stable subsequent treatment through medications in the residing country after receiving the telemedicine service.

### CROSS-REFERENCE TO RELATED APPLICATION

This application is based on and claims priority under 35 U.S.C. § 119 to Korean Patent Application No. 10-2020-0087384, filed on Jul. 15, 2020, in the Korean Intellectual Property Office, the disclosure of which is incorporated by reference herein in its entirety.

### BACKGROUND

### 1. Field

The present disclosure relates to a global telemedicine system for issuing a country-customized prescription, and more particularly, to a medication analyzing and matching system capable of generating a prescription using a language and a replaceable medication of a telemedicine service receiving county, based on a prescription prescribed by using a language and medication of a telemedicine service providing county during telemedicine between different countries, and issuing the generated prescription to a patient, a global telemedicine system including such a system, and a method of generating such a prescription for different countries.

### 2. Description of Related Art

Recently, demands for the spread of untact treatment systems have been rapidly increased due to issues, such as the spread of virus infection and the collapse of medical systems, and a telemedicine service system is emerging as a solution. Such a telemedicine service includes providing of services, such as biometric information monitoring, guideline on health, a video consultation, and prescription issue, to a user.

In the global era, cases where patients who live in countries with insufficient medical facilities or a low level of medical staffs visit a medical service system of an advanced country with relatively abundant medical resources and a high degree of infrastructure have explosively increased, but due to the recent spread of virus infection, the borders between countries are closed or visits between countries became not easy, and thus demands for a global telemedicine service system are rapidly increasing. In the case of Republic of Korea, Korean nationals residing broad permanently or temporarily suffer from difficulty in using a hospital in the overseas residence due to a problem, such as insurance, medical expenses, or language, and thus a telemedicine service targeting the Korean nationals residing broad is also actively carried forward.

However, one of main problems of the telemedicine service between a doctor and a patient in different countries is that a prescription including medications prescribed by the doctor in the country providing the telemedicine service is often unable to be used in the other country where the patient resides, because the classification standards for distributed ethical (ETC) and over-the-counter (OTC) medications are different, and types, names, ingredients, contents, dosage regimen, allowed additives, and the like of medications or prescription medicines are different between the country providing the telemedicine service and the country receiving the telemedicine service.

In this regard, the country providing the telemedicine service may purchase the medications according to the prescription and deliver the medications to the patient in the other country, but if there is no agreement between the countries, the prescribed medicines are mostly confiscated and discarded by foreign customs, and even if the prescribed medicines are cleared, the patient may receive the prescribed medicines after a fairly long period of time due to overseas delivery. Thus, treatment through the medications of the prescription issued via the telemedicine service is often not useful to the patient.

In addition, even if the patient is to purchase the medications of the prescription in the language of the country providing the telemedicine service or provide the prescription to a local doctor for reference medical information, a local pharmacist or medical staff may not understand the prescription.

### SUMMARY

The present disclosure enables a patient to receive appropriate subsequent treatment via alternative medications in a residing country after receiving a telemedicine service by providing, to the patient, a prescription localized to the residing county of the patient receiving the telemedicine service, based on a prescription issued by a hospital or doctor of a country providing the telemedicine service.

Also, the present disclosure enables a patient to easily obtain alternative medications in his/her own country or facilitates cooperative treatment or additional subsequent treatment in a residing county by automatically translating a prescription issued in a language of a country providing a telemedicine service to a language available in the residing country of the patient receiving the telemedicine service and providing the prescription such that not only the patient but also a medical staff or pharmacist of the residing county of the patient understands the prescription.

Also, the present disclosure enables a patient to easily obtain, from a residing country, medications that best correspond to ethical (ETC) medications of a prescription issued by a doctor in a country providing a telemedicine service, even when medication items classified as ETC medications that require a prescription and over-the-counter (OTC) medications purchasable without a prescription are different between the country of the hospital or doctor providing the telemedicine service and the residing county of the patient receiving the telemedicine service.

Additional aspects will be set forth in part in the description which follows and, in part, will be apparent from the description, or may be learned by practice of the presented embodiments of the disclosure.

A global medication analyzing and matching system according to an embodiment of the present disclosure includes:

a receiving unit configured to receive, from a telemedicine service providing hospital of a first country, a first country language prescription including medications of the first country prescribed for a patient of a second country;

a medication analyzing unit configured to analyze medication detail information including ingredients, contents, effects, dosage regimen, and additives for each of medication items in the first country language prescription, based on received medication database information of the first country;

a medication matching unit configured to search a medication database of the second country for each of the medication items based on the analyzed medication detail information to calculate matching degrees of each of the medication items with found medications in the second country, and generate a second country prescription by using, as a second country alternative medication, a medication of the second country having a highest value of matching degree for each of the medication items;

a language converting unit configured to generate a second country language prescription by converting a language of the second country prescription from a first country language to a second country language;

a transmitting unit configured to transmit the second country language prescription to the patient in the second country; and

a database storing data including the first country language prescription, the second country prescription, and the second country language prescription.

The matching degrees may be calculated by calculating similarity between each of the medication items of the first country language prescription and the medications of the second country with respect to the medication detail information including the ingredients, contents, effects, dosage regimen, and additives, according to a matching algorithm of the medication matching unit, wherein a pre-determined individual weight may be assigned to each item in the medication detail information.

The medication matching unit may be further configured to:

calculate matching degrees of each of the medication items with ethical medications of an ethical medication database of the second country, based on the analyzed medication detail information, and use, as the second country alternative medication, a second country ethical medication having a highest value of matching degree from among second country ethical medications having a certain value of matching degree or higher; and

when the second country ethical medications having the certain value of matching degree or higher do not exist, calculate matching degrees of each of the medication items with over-the-counter medications of an over-the-counter medication database of the second country, based on the analyzed medication detail information, and use, as the second country alternative medication, a second country over-the-counter medication having a highest value of matching degree from among second country over-the-counter medications having the certain value of matching degree or higher.

The medication matching unit may be further configured to provide detail information of medication items of the second country prescription to the telemedicine service providing hospital of the first country after the second country prescription is generated such that suitability of the second country prescription is verified and the telemedicine service providing hospital of the first country manually revises the second country prescription when necessary.

The medication matching unit may be further configured to:

calculate matching degrees of each of the medication items with ethical medications of an ethical medication database of the second country and matching degrees of each of the medication items with over-the-counter medications of an over-the-counter medication database of the second country, based on the analyzed medication detail information, select ethical medications and over-the-counter medications of the second country having a certain value of matching degree or higher as second country alternative medication candidate group, and generate the second country prescription by using, as the second country alternative medication, a medication having a highest value of matching degree in the second country alternative medication candidate group; and

provide detail information of medication items of the second country prescription and detail information of the second country alternative medication candidate group to the telemedicine service providing hospital of the first country after the second country prescription is generated such that suitability of the second country prescription is verified and the telemedicine service providing hospital of the first country manually revises the second country prescription based on the detail information of the second country alternative medication candidate group and treatment information of the patient of the second country when necessary.

The medication matching unit may be further configured to, when selecting the second country alternative medication candidate group, exclude an ethical medication or over-the-counter medication of the second country containing a drug-sensitive ingredient for the patient of the second country.

The database may additionally store data including the second country alternative medication candidate group and the second country prescription revised by the telemedicine service providing hospital of the first country, and the global medication analyzing and matching system may further include a matching model updating unit configured to update a matching algorithm for calculating the matching degrees by learning the data stored in the database.

The transmitting unit may be further configured to transmit the second country language prescription to a cooperative hospital of the second country.

A global telemedicine service system for providing a telemedicine service between a telemedicine service providing hospital of a first country and a patient of a second country, according to another embodiment of the present disclosure, includes:

a telemedicine communication unit configured to transmit and receive data such that the telemedicine service is providable between the telemedicine service providing hospital of the first country and the patient of the second country;

a prescription analyzing/matching unit configured to analyze, based on medication database information of the first country, medication detail information including ingredients, contents, effects, dosage regimen, and additives of each of medication items of a first country language prescription including medications received from the telemedicine service providing hospital via the telemedicine communication unit and issued for the patient, calculate matching degrees of each of the medication items with medications of a medication database of the second country, based on the analyzed medication detail information, and generate a second country prescription by using, as a second country alternative medication, a medication of the second country having a highest value of matching degree for each of the medication items;

a language converting unit configured to convert data in a first country language to a second country language and provide the same to the patient, and generate a second country language prescription by converting a language of the second country prescription from the first country language to the second country language, while the telemedicine service is provided via the global telemedicine system; and

a database storing data including data generated when the telemedicine service is provided, information related to the first country language prescription, information related to the second country prescription, and information related to the second country language prescription,

wherein the telemedicine communication unit is further configured to transmit the second country language prescription to the patient of the second country.

The matching degrees may be calculated by calculating, by the prescription analyzing/matching unit, similarity of each item of the medication detail information including ingredients, contents, effects, dosage regimen, and additives,

wherein a pre-determined individual weight may be assigned to each item of the medication detail information.

The prescription analyzing/matching unit may include:

a medication analyzing module configured to analyze the medication detail information including ingredients, contents, effects, dosage regimen, and additives for each of medication items in the first country language prescription, based on the medication database information of the first country; and

a medication matching module configured to calculate the matching degrees of each of the medication items with the medications of the medication database of the second country, based on the analyzed medication detail information, and generate the second country prescription by using, as the second country alternative medication, the medication of the second country having the highest value of matching degree for each of the medication items.

The medication matching module may be further configured to:

calculate matching degrees of each of the medication items with ethical medications of an ethical medication database of the second country, based on the analyzed medication detail information, and use, as the second country alternative medication, a second country ethical medication having a highest value of matching degree from among second country ethical medications having a certain value of matching degree or higher; and

when the second country ethical medications having the certain value of matching degree or higher do not exist, calculate matching degrees of each of the medication items with over-the-counter medications of an over-the-counter medication database of the second country, based on the analyzed medication detail information, and use, as the second country alternative medication, a second country over-the-counter medication having a highest value of matching degree from among second country over-the-counter medications having the certain value of matching degree or higher.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a schematic view of a medication analyzing and matching system used to issue a localized prescription of a telemedicine service, according to an embodiment of the present disclosure;

FIG. 1B is a block diagram of a medication analyzing and matching system according to an embodiment of the present disclosure;

FIG. 2 is a block diagram of a global telemedicine system according to an embodiment of the present disclosure;

FIG. 3 is a block diagram of a prescription analyzing/matching unit of FIG. 2;

FIG. 4 is a flowchart of a method of performing a country-customized prescription providing service to a patient in a global telemedicine service, according to an embodiment of the present disclosure;

FIG. 5 is a flowchart of a method of performing a country-customized prescription providing service to a patient in a global telemedicine service, according to another embodiment of the present disclosure; and

FIG. 6 is a flowchart of a method of performing a country-customized prescription providing service to a patient in a global telemedicine service, according to another embodiment of the present disclosure.

### DETAILED DESCRIPTION

Hereinafter, the present disclosure will be described with reference to accompanying drawings. However, the present disclosure may be implemented in various different forms and is not limited to embodiments described herein. Also, in the drawings, parts irrelevant to the description are omitted in order to clearly describe the present disclosure, and like reference numerals designate like elements throughout the specification.

Throughout the specification, when a part is “connected to (in contact with or combined to)” another part, the part may not only be “directly connected to” the other part, but may also be “indirectly connected to” the other part with another member in between. In addition, when a part “includes” a certain element, the part may further include another element instead of excluding the other element, unless otherwise stated.

Also, the terms used in the present specification are only used to describe specific embodiments, and are not intended to limit the present disclosure. An expression used in the singular encompasses the expression in the plural, unless it has a clearly different meaning in the context. In the present specification, it is to be understood that terms such as “including” or “having”, etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Hereinafter, exemplary embodiments are presented for understanding of the present disclosure, but the embodiments are only examples and it would be obvious to one of ordinary skill in the art that various changes and modifications are possible within the scope and spirit of the present disclosure. Further, it would be obvious that such changes and modifications belong to the appended claims.

Hereinafter, embodiments of the present disclosure will be described in detail with reference to accompanying drawings.

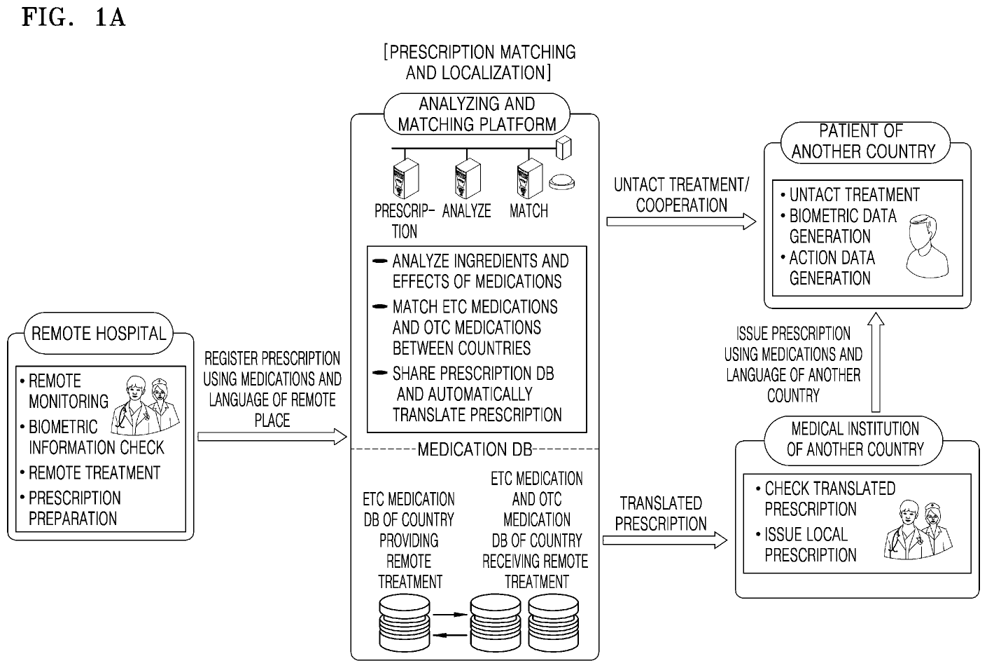

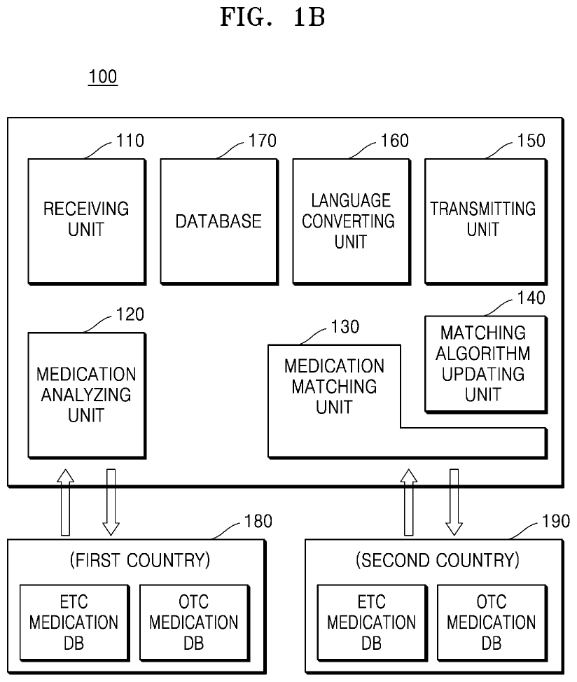

FIG. 1A is a schematic view of a global telemedicine service in which a medication analyzing and matching system issues a localized prescription of a telemedicine service, according to an embodiment of the present disclosure.

Referring to FIG. 1A, when a hospital or doctor providing a telemedicine service and a patient are in different countries, the doctor providing the telemedicine service may perform telemedicine by obtaining information about the patient via a method of remotely monitoring biometric data or action data of the patient in another country using a sensor or medical devices connected to the patient via a network, such as the Internet, receiving past medical record information of the patient from the patient or an existing hospital in charge of the patient and examining the past medical record information, or interviewing the patient through a real-time untact consultation. After such telemedicine, the doctor may issue a prescription such that the patient receives an appropriate subsequent drug treatment according to a diagnosis. Such a prescription is prepared in a language commonly used in a country of the hospital or doctor providing the telemedicine service and includes items of medications approved in the corresponding country.

The present disclosure is derived based on a fact that there are many cases where the predication including the medication prescribed by the doctor of the country providing the telemedicine service is unable to be used in the other country where the patient resides, because classification standards for distributed ethical (ETC) medications and over-the-counter (OTC) medications are different, and types, names, ingredients, contents, dosage regimen, and allowed additives of medication or prescription medicines are different between the country providing the telemedicine service and the country receiving the telemedicine service.

The medication analyzing and matching system according to an embodiment of the present disclosure provides a country-customized prescription to the patient of the other country, by analyzing detail information of ingredients and effects of medications included in an original prescription issued by the doctor, generating a localized prescription usable in the other county of the patient by matching the medications included in the original prescription with medications of the other country where the patient resides, and automatically translating the localized prescription to a language commonly used in the other country of the patient. The medication analyzing and matching system may analyze the detail information of ingredients and effects by receiving medication-related information included in the original prescription in association with a medication database (DB) of the country providing the telemedicine service. For example, when the country providing the telemedicine service is Republic of Korea, the medication DB may be an ETC and/or OTC medication database provided by the Ministry of Food and Drug Safety or a medication database provided by the Korea Pharmaceutical Information Center.

Also, when the classification standards of distributed ETC medications and OTC medications are different between the country providing the telemedicine service and the country receiving the telemedicine service, the medication analyzing and matching system of FIG. 1A may analyze the ingredients and effects of the medications in the original prescription, first search for ETC medications having highest matching degrees with the medications in the original prescription in relation to the analyzed ingredients and effects, based on ETC medication information of an ETC medication DB of the country where the patient resides, and use the ETC medications having the highest matching degrees as alternative medications of the localized prescription. When it is unable to derive alternative medications having matching degrees equal to or greater than a certain value or level from the ETC medication DB of the country where the patient resides, the medication analyzing and matching system may search for alternative OTC medications having highest matching degrees with the medications in the original prescription in relation to the analyzed ingredients and effects, based on OTC medication information of an OTC medication DB of the country where the patient resides, and use the OTC medications having the highest matching degrees as the alternative medications of the localized prescription.

The prescription localized by matching the medications of the original prescription to the alternative medications of the country where the patient resides as such may be finally issued to the patient after being automatically translated in the language commonly used in the country where the patient resides.

FIG. 1B is a block diagram of a medication analyzing and matching system 100 according to an embodiment of the present disclosure, which may be used in the global telemedicine service of FIG. 1A between a telemedicine service providing hospital or doctor of a first country and a patient of a second country.

Referring to FIG. 1B, the medication analyzing and matching system 100 according to an embodiment of the present disclosure may include a receiving unit 110, a medication analyzing unit 120, a medication matching unit 130, a transmitting unit 150, a language converting unit 160, and a DB 170. The medication analyzing and matching system 100 may further include a matching algorithm updating unit 140.

The medication analyzing and matching system 100 may be connected to a first-country medication DB 180 to search and request for medication-related detail information, and the first-country medication DB 180 may include an ETC medication DB and/or an OTC medication DB of the first country. The medication analyzing and matching system 100 may also be connected to a second-country medication DB 190 to search and request for medication-related detail information, and the second-country medication DB 190 may include an ETC medication DB and/or an OTC medication DB of the second country.

Medication lists and related information received from the first-country medication DB 180 and the second-country medication DB 190 may be reclassified into detail information items including following items and stored in the DB 170.

(a) Manufacturing Country and/or Selling Country

(b) Ingredient Name and/or Ingredient Content

(c) Additive Name

(d) Efficacy and Effect

(e) Dosage regimen

(f) ETC Medication/OTC Medication

(g) Drug/Quasi-Drug

(h) Current Availability of Supply in Corresponding Country

(i) Matching Code

At least one of the detail information items above may be used to determine similarity according to a matching algorithm while matching medication information of the first-country medication DB 180 and medication information of the second-country medication DB 190.

The receiving unit 110 may receive a first country language prescription including medications of the first country prescribed for the patient of the second country as a result of telemedicine by the telemedicine service providing hospital or doctor of the first country. Also, the receiving unit 110 may receive medication-related information from the first-country medication DB 180 and/or the second-country medication DB 190.

The medication analyzing unit 120 may analyze medication detail information including ingredients, effects, dosage regimen, and additives for each medication item in the first country language prescription, based on information received from the first-country medication DB 180 via the receiving unit 110. A matching code may be assigned to a medication of which the medication detail information is analyzed.

The medication matching unit 130 may search the second-country medication DB 190 where the patient resides and calculate matching degrees of each medication item with found medications of the second country, based on the analyzed medication detail information, assign, to a medication of the second country having a highest value of matching degree for each medication item, a matching code corresponding to a matching code of the medication of the first country language prescription, and generate a second country prescription using the medication of the second country having the highest value of matching degree as a second country alternative medication.

For example, when the doctor of the first country prescribed a digestive medicine a in the first country language prescription, the medication analyzing unit 120 may search for and receive medication detail information including ingredients, contents, effects, dosage regimen, and additives of the digestive medicine a from data of the first-country medication DB 180, analyze the medication detail information, and assign a matching code (for example, a matching code A1) indicating a corresponding country and medication to the digestive medicine a. Then, the medication matching unit 130 may search the data of the second-country medication DB 190 for digestive medicines having the same or similar ingredients, contents, effects, dosage regimen, and additives as the digestive medicine a, calculate matching degrees of the digestive medicine a with found digestive medicines, based on the analyzed medication detail information of the digestive medicine a, and assign a corresponding matching code (for example, a matching code B1) to a digestive medicine b having a highest value of matching degree. Accordingly, when a doctor prepares a prescription with a medication of the matching code A1, the medication matching unit 130 may be programmed to list similar medications to which the corresponding matching code B1 of the second country is assigned.

The medication matching unit 130 may be further configured to, in addition to selecting a medication of a corresponding matching code, list similar product groups even when related information, such as a manufacturing and selling country, product name, ingredient name, efficacy, effect, dosage regimen, additive of a medication, is found from the first-country medication DB 180 providing the telemedicine.

The matching degree may be calculated by calculating similarity between each medication item of the first country language prescription and a medication of the second country, based on the medication detail information, such as ingredients, effects, dosage regimen, according to the matching algorithm of the medication matching unit 130. While calculating the matching degree, a pre-determined individual weight may be assigned to each item of the medication detail information. The matching algorithm may be a matching algorithm by an artificial intelligence (AI), and at this time, the individual weight may be trained and derived via machine learning or deep learning based on data from the DB 170 or an external DB (for example, the medication DB 180 or the medication DB 190).

The medication matching unit 130 may provide detail information of medication items of the second country prescription to the telemedicine service providing hospital of the first country after the second country prescription is generated such that suitability of the second country prescription is verified and the telemedicine service providing hospital of the first country may manually revise the second country prescription when necessary to confirm the second country prescription for the patient.

According to another embodiment of the present disclosure, the medication matching unit 130 may calculate matching degrees of the each medication item of the first country language prescription with ETC medications of the ETC medication DB of the second country, based on the analyzed medication detail information, and generate the second country prescription by using, as the second country alternative medication, a second country ETC medication having a highest value of matching degree from among second country ETC medications having a certain value of matching degree or higher. When the second country ETC medications having the certain value of matching degree or higher do not exist, the medication matching unit 130 may calculate matching degrees of the each medication item with OTC medications of the OTC medication DB of the second country, based on the analyzed medication detail information, and generate the second country prescription by using, as the second country alternative medication, a second country OTC medication having a highest value of matching degree from among second country OTC medications having the certain value of matching degree or higher.

According to another embodiment of the present disclosure, the medication matching unit 130 may calculate matching degrees of the each medication item of the first country language prescription with the ETC medications of the ETC medication DB of the second country and the OTC medications of the OTC medication DB of the second country, based on the analyzed medication information, select second country ETC medications and OTC medications having a certain value of matching degree or higher as a second country alternative medication candidate group, and generate the second country prescription by using, as the second country alternative medication, a medication having a highest value of matching degree in the second country alternative medication candidate group. After the second country prescription is generated, the medication matching unit 130 may provide, to the telemedicine service providing hospital of the first country, detail information of medication items of the second country prescription and detail information of the second country alternative medication candidate group such that suitability of the second country prescription is verified, and the telemedicine service providing hospital of the first country may manually revise the second country prescription based on the detail information of second country alternative medication candidate group and treatment information of the patient of the second country when necessary to confirm the second country prescription.

The medication matching unit 130 may, when selecting the second country alternative medication candidate group, exclude an ETC medication or OTC medication of the second country containing a drug-sensitive ingredient for the patient of the second country.

While generating the second country prescription, the medication matching unit 130 may consider information about availability of supply of medications in the second country, which is stored in the second-country medication DB 190 or the DB 170, to exclude a medication that is impossible to be supplied in the second country when such a medication has a highest value of matching degree with a medication of an original prescription, and generate the second country prescription by replacing the medication that is impossible to be supplied with a similar medication having a highest value of matching degree from among other similar medications having values of matching degree lower than the highest value of matching degree of the medication that is impossible to be supplied.

The language converting unit 160 may generate a second country language prescription by converting a language of the second country prescription from a first country language to a second country language. The language converting unit 160 may automatically translate voice or input text of the doctor of the first country and/or the patient of the second country during telemedicine between the doctor and the patient to display translated text on a terminal of the doctor and/or patient or utter the translated voice via a chatbot, such that medication counseling and guidance of the telemedicine or prescription are smoothly understood. The language converting unit 160 may learn existing automatic translation results of telemedicine consultations and prescriptions via machine learning and/or deep learning to update a language converting algorithm used for automatic translation.

The DB 170 may include the first country language prescription, the second country prescription, and the second country language prescription. The DB 170 may additionally store the second country alternative medication candidate group, the second country prescription revised by the telemedicine service providing hospital of the first country, and related detail information. The DB 170 may store lists of matching codes and similar medications generated by the medication analyzing unit 120 and the medication matching unit 130. The DB 170 may additionally store data required for the telemedicine service, such as basic information and biometric information of the patient, medication information, medical terms, and medical information.

The DB 170 may receive a medication list and related detail information of the first country from the first-country medication DB 180, and store the same. The DB 170 may receive a medication list and related detail information of the second country from the second-country medication DB 190, and store the same. The DB 170 may receive the first country language prescription for the patient together with drug- or food-sensitive information of the patient from the doctor, and store the same. When medication DB of each country is updated, the DB 170 receives the updated DB to prepare for a case when similar medications are difficult to be supplied in the second country, and further stores information about availability of supply for each product in a medication list of each country such as to be used by the medication matching unit 130 while preparing the second country prescription.

The matching algorithm updating unit 140 may update the matching algorithm for calculating the matching degrees by learning the data stored in the DB 170 via machine learning and/or deep learning. The updated matching algorithm is used by the medication matching unit 130 again to derive the second country prescription.

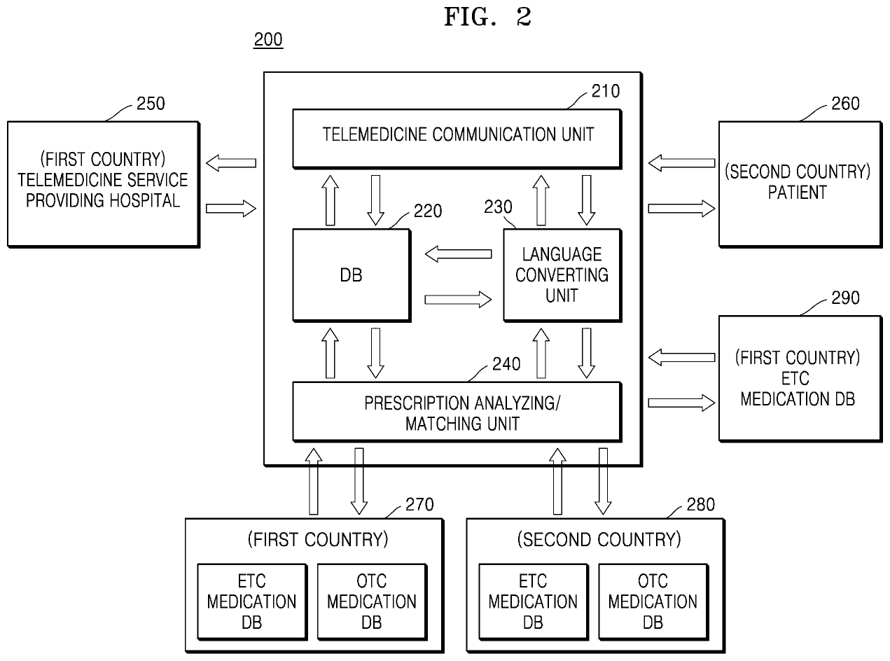

FIG. 2 is a block diagram of a global telemedicine system 200 according to an embodiment of the present disclosure.

The global telemedicine system 200 according to an embodiment of the present disclosure may be a platformized system enabling various telemedicine services between a first-country telemedicine service providing hospital 250 or a doctor and a second-country patient 260. A second-country cooperative hospital 290 or a doctor may also access the global telemedicine system 200 for the second-country patient 260 to provide, simultaneously or non-simultaneously with telemedicine, cooperative treatment or a service, such as sharing of a medical record and prescription of the second country patient 260. The global telemedicine system 200 may perform the telemedicine by obtaining information about the second country patient 260 via a method of remotely monitoring biometric data or action data of the second-country patient 260 via medical devices such as a sensor connected to the patient 260 through a network, such as the Internet, a method of receiving past medical record information of the second country patient 260 from the second country patient 260 or an existing hospital or doctor in charge of the second country patient 260 and examining the past medical record information, or a method of interviewing the second country patient 260 via a real-time untact consultation.

Referring to FIG. 2, the global telemedicine system 200 may include a telemedicine communication unit 210, a DB 220, a language converting unit 230, and a prescription analyzing/matching unit 240. The global telemedicine system 200 may be connected to a first-country medication DB 270 to search and request for medication-related detail information, and the first-country medication DB 270 may include an ETC medication DB and/or an OTC medication DB of the first country. The global telemedicine system 200 may also be connected to a second-country medication DB 280 of to search and request for medication-related detail information, and the second-country medication DB 280 of may include an ETC medication DB and/or an OTC medication DB of the second country.

Medication lists and related information received from the first-country medication DB 270 and the second-country medication DB 280 may be reclassified into detail information items including following items and stored in the DB 220.

(a) Manufacturing Country and/or Selling Country

(b) Ingredient Name and/or Ingredient Content

(c) Additive Name

(d) Efficacy and Effect

(e) Dosage regimen

(f) ETC Medication/OTC Medication

(g) Drug/Quasi-Drug

(h) Current Availability of Supply in Corresponding Country

(i) Matching Code

At least one of the detail information items above may be used to determine similarity according to a matching algorithm while matching medication information of the first-country medication DB 270 and medication information of the second-country medication DB 280.

The telemedicine communication unit 210 is configured to transmit or receive data, image, or the like to or from an external entity via a network, and to enable the first-country telemedicine service providing hospital 250 or a doctor, the second-country patient 260, and/or the second-country cooperative hospital 290 or a doctor to exchange and share telemedicine, consultation, and information. Also, the telemedicine communication unit 210 is configured to access the first-country medication DB 270 and/or the second-country medication DB 280 of to request or search for data or receive data therefrom. Accordingly, the telemedicine communication unit 210 may receive a first country language prescription including medications of the first country prescribed for the second-country patient 260 as a result of the telemedicine.

The DB 220 may include the first country language prescription, a second country prescription, and a second country language prescription. The DB 220 may additionally store a second country alternative medication candidate group, the second country prescription revised by the first-country telemedicine service providing hospital 250, and related detail information. The DB 220 may store lists of matching codes and similar medications generated by the DB 220.

The DB 220 may receive a medication list and related detail information of the first country from the first-country medication DB 270, and store the same. The DB 220 may receive a medication list and related detail information of the second country from the second-country medication DB 280 of, and store the same. The DB 220 may receive the first country language prescription for the patient 260 together with drug- or food-sensitive information of the patient 260 from the first-country telemedicine service providing hospital 250 or doctor, and store the same. When the first-country medication DB 270 and the second-country medication DB 280 are updated, the DB 220 receives the updated DBs to prepare for a case when similar medications are difficult to be supplied in the second country, and further stores information about availability of supply for each product in a medication list of each country such as to be used by the prescription analyzing/matching unit 240 while deriving the second country prescription after calculating matching degrees.

The language converting unit 230 may generate the second country language prescription by converting a language of the second country prescription from a first country language to a second country language, and automatically translate voice or input text of the doctor of the first country and/or the second-country patient 260 during the telemedicine between the doctor of the first country and the second-country patient 260 to display the translated text on a terminal of the doctor and/or patient 260 or utter the translated voice via a chatbot, such that medication counseling and guidance of the telemedicine or prescription are smoothly understood. The language converting unit 230 may learn existing automatic translation results of telemedicine consultations and prescriptions via machine learning and/or deep learning to update a language converting algorithm used for automatic translation.

The prescription analyzing/matching unit 240 analyzes, based on medication DB information of the first country, medication detail information including ingredients, contents, effects, dosage regimen, and additives of each of medication items of the first country language prescription issued for the second-country patient 260 and received from the first-country telemedicine service providing hospital 250 via the telemedicine communication unit 210, calculate matching degrees of each of the medication items with medications of the second-country medication DB 280 of, based on the analyzed medication detail information, and generate the second country prescription by using, as a second country alternative medication, a medication of the second country having a highest value of matching degree for each of the medication items.

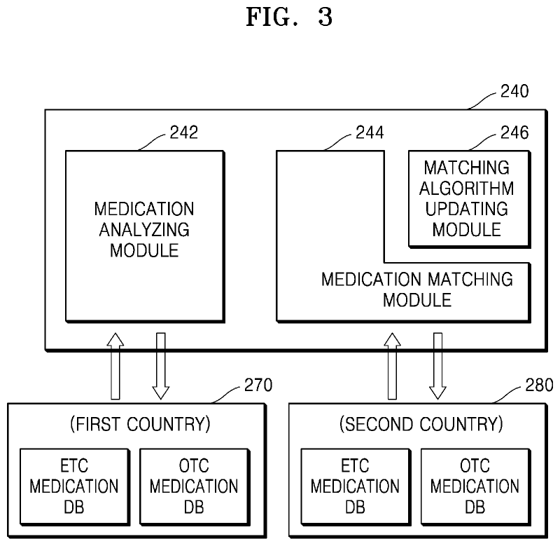

FIG. 3 is a block diagram of the prescription analyzing/matching unit 240 of FIG. 2.

Referring to FIG. 3, the prescription analyzing/matching unit 240 may include a medication analyzing module 242 and a medication matching module 244, and may further include a matching algorithm updating module 246.

The medication analyzing module 242 and the medication matching module 244 of FIG. 3 may include all configurations corresponding to functions of the medication analyzing unit 120 and the medication matching unit 130 of the medication analyzing and matching system 100 of FIG. 1B.

In particular, the medication analyzing module 242 may analyze medication detail information including ingredients, effects, dosage regimen, and additives for each medication item in the first country language prescription, based on information received from the first-country medication DB 270 via the telemedicine communication unit 210. A matching code may be assigned to a medication of which the medication detail information is analyzed.

The medication matching module 244 may search the second-country medication DB 280 of for each medication item of the first country language prescription, based on the analyzed medication detail information, calculate matching degrees of the each medication item with found medications of the second country, and generate the second country prescription by using, as a second country alternative medication, a medication of the second country having a highest value of matching degree for the each medication item.

The matching degree may be calculated by calculating similarity between each medication of the first country language prescription and a medication of the second country, based on the medication detail information, such as ingredients, effects, dosage regimen, and additives, according to the matching algorithm of the medication analyzing module 242. While calculating the matching degree, a pre-determined individual weight may be assigned to each item of the medication detail information. The matching algorithm may be a matching algorithm by AI, and at this time, the individual weight may be trained via machine learning or deep learning based on data from the DB 220 or an external DB (for example, the first-country medication DB 270 or the second-country medication DB 280).

The medication matching module 244 may provide detail information of medication items of the second country prescription to the first-country telemedicine service providing hospital 250 after the second country prescription is generated such that suitability of the second country prescription is verified and the first-country telemedicine service providing hospital 250 may manually revise the second country prescription when necessary to confirm the second country prescription for the patient 260.

For example, when the doctor of the first country prescribed a digestive medicine a in the first country language prescription, the medication analyzing module 242 may search for and receive medication detail information including ingredients, contents, effects, dosage regimen, and additives of the digestive medicine a from data of the first-country medication DB 270, analyze the medication detail information, and assign a matching code (for example, a matching code A1) indicating a corresponding country and medication to the digestive medicine a. Then, the medication matching module 244 may search the data of the second-country medication DB 280 of for digestive medicines having the same or similar ingredients, contents, effects, dosage regimen, and additives as the digestive medicine a, calculate matching degrees of the digestive medicine a with found digestive medicines, based on the analyzed medication detail information of the digestive medicine a, and assign a corresponding matching code (for example, a matching code B1) to a digestive medicine b having a highest value of matching degree. Accordingly, when a doctor prepares a prescription with a medication of the matching code A1, the medication matching module 244 may be programmed to list similar medications to which the corresponding matching code B1 of the second country is assigned.

The medication matching module 244 may be further configured to, in addition to selecting a medication of a corresponding matching code, list similar product groups even when related information, such as a manufacturing or selling country, product name, ingredient name, efficacy, effect, dosage regimen, additive of a medication, is found from the first-country medication DB 270 providing the telemedicine.

The matching degree may be calculated by calculating similarity between each medication item of the first country language prescription and a medication of the second country, based on the medication detail information, such as ingredients, effects, dosage regimen, according to the matching algorithm of the medication matching module 244. While calculating the matching degree, a pre-determined individual weight may be assigned to each item of the medication detail information. The matching algorithm may be a matching algorithm by AI, and at this time, the individual weight may be trained via machine learning or deep learning based on data from the DB 220 or an external DB (for example, the first-country medication DB 270 or the second-country medication DB 280).

The medication matching module 244 may provide detail information of medication items of the second country prescription to the first-country telemedicine service providing hospital 250 after the second country prescription is generated such that suitability of the second country prescription is verified and the first-country telemedicine service providing hospital 250 may manually revise the second country prescription when necessary to confirm the second country prescription for the patient 260.

According to another embodiment of the present disclosure, the medication matching module 244 may calculate matching degrees of the each medication item of the first country language prescription with ETC medications of the ETC medication DB of the second country, based on the analyzed medication detail information, and generate the second country prescription by using, as the second country alternative medication, a second country ETC medication having a highest value of matching degree from among second country ETC medications having a certain value of matching degree or higher. When the second country ETC medications having the certain value of matching degree or higher do not exist, the medication matching module 244 may calculate matching degrees of the each medication item with OTC medications of the OTC medication DB of the second country, based on the analyzed medication detail information, and generate the second country prescription by using, as the second country alternative medication, a second country OTC medication having a highest value of matching degree from among second country OTC medications having the certain value of matching degree or higher.

According to another embodiment of the present disclosure, the medication matching module 244 may calculate matching degrees of the each medication item of the first country language prescription with the ETC medications of the ETC medication DB of the second country and the OTC medications of the OTC medication DB of the second country, based on the analyzed medication information, select second country ETC medications and OTC medications having a certain value of matching degree or higher as a second country alternative medication candidate group, and generate the second country prescription by using, as the second country alternative medication, a medication having a highest value of matching degree in the second country alternative medication candidate group. After the second country prescription is generated, the medication matching module 244 may provide, to the telemedicine service providing hospital 250 of the first country, detail information of medication items of the second country prescription and detail information of the second country alternative medication candidate group such that suitability of the second country prescription is verified, and the first-country telemedicine service providing hospital 250 may manually revise the second country prescription based on the detail information of second country alternative medication candidate group and treatment information of the second-country patient 260 when necessary to confirm the second country prescription.

The medication matching module 244 may, when selecting the second country alternative medication candidate group, exclude an ETC medication or OTC medication of the second country containing a drug-sensitive ingredient for the second-country patient 260.

While generating the second country prescription, the medication matching module 244 may consider information about availability of supply of medications in the second country, which is stored in the second-country medication DB 280 or the DB 220, to exclude a medication that is impossible to be supplied in the second country when such a medication has a highest value of matching degree with a medication of an original prescription, and generate the second country prescription by replacing the medication that is impossible to be supplied with a similar medication having a highest value of matching degree from among other similar medications having values of matching degree lower than the highest value of matching degree of the medication that is impossible to be supplied.

The matching algorithm updating module 246 may update the matching algorithm for calculating the matching degrees by learning the data stored in the DB 220 via machine learning and/or deep learning. The updated matching algorithm is used by the medication matching module 244 again to derive the second country prescription.

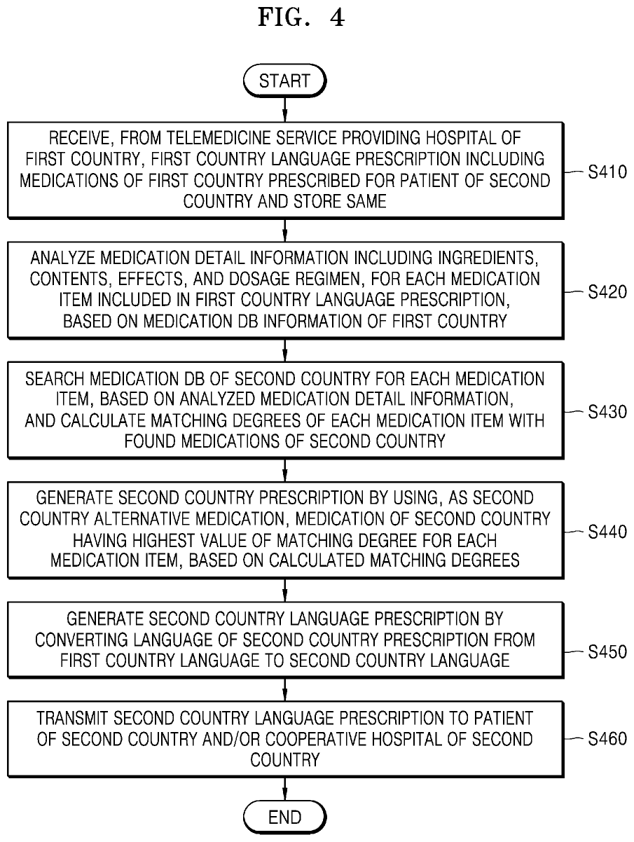

FIG. 4 is a flowchart of a method of performing a country-customized prescription providing service to a patient in a global telemedicine service, according to an embodiment of the present disclosure. The method may be performed by the medication analyzing and matching system 100 of FIGS. 1A and 1B or the global telemedicine system 200 of FIG. 2, according to an embodiment of the present disclosure.

Referring to FIG. 4, in the method according to an embodiment of the present disclosure, the global telemedicine system 200 receives, from the first-country telemedicine service providing hospital 250, the first country language prescription including the medications of the first country prescribed for the second-country patient 260, and stores the same (operation S410). The global telemedicine system 200 analyzes the medication detail information including ingredients, contents, effects, dosage regimen, and additives for each medication item included in the first country language prescription, based on the medication DB information of the first country stored in a connected external institute or an internal DB (operation S420). Then, the global telemedicine system 200 searches the medication DB of the second country for each medication item of the first country language prescription, based on the analyzed medication detail information, and calculates the matching degrees of each medication item with the found medications of the second country (operation S430). The global telemedicine system 200 generates the second country prescription by using, as the second country alternative medication, the medication of the second country having the highest value of matching degree for each medication item of the first country language prescription, based on the calculated matching degrees (operation S440). Then, the global telemedicine system 200 generates the second country language prescription by converting or automatically translating the language of the generated second country prescription from the first country language to a language commonly used in the second country (operation S450). The second country language prescription generated as such is transmitted to the second-country patient 260 and/or the second-country cooperative hospital 290 (operation S460).

According to another embodiment of the present disclosure, the global telemedicine system 200 may provide, between operations S440 and S450, the detail information of the medication item of the second country prescription to the first-country telemedicine service providing hospital 250 after the second country prescription is generated such that the suitability of the second country prescription is verified, and the first-country telemedicine service providing hospital 250 may manually revise the second country prescription to confirm the second country prescription for the second-country patient 260 when necessary. At this time, the first-country telemedicine service providing hospital 250 may exclude or revise a medication of the second country prescription, based on medication information, such as drug-sensitive information of the second-country patient 260.

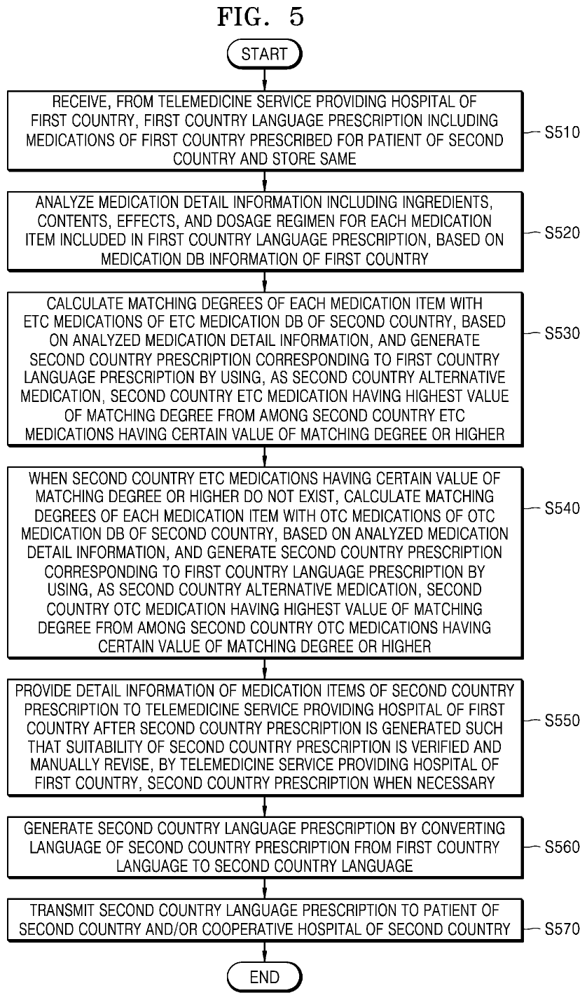

FIG. 5 is a flowchart of a method of performing a country-customized prescription providing service to a patient in a global telemedicine service, according to another embodiment of the present disclosure. The method may be performed by the medication analyzing and matching system 100 of FIGS. 1A and 1B or the global telemedicine system 200 of FIG. 2, according to an embodiment of the present disclosure.

Referring to FIG. 5, in the method according to an embodiment of the present disclosure, the global telemedicine system 200 receives, from the first-country telemedicine service providing hospital 250, the first country language prescription including the medications of the first country prescribed for the second-country patient 260, and stores the same (operation S510). The global telemedicine system 200 analyzes the medication detail information including ingredients, contents, effects, dosage regimen, and additives for each medication item included in the first country language prescription, based on the medication DB information of the first country stored in a connected external institute or an internal DB (operation S520). Then, the global telemedicine system 200 calculates the matching degrees of each medication items with the ETC medications of the ETC medication DB of the second country, based on the analyzed medication detail information, and generates the second country prescription corresponding the first country language prescription by using, as the second country alternative medication, the second country ETC medication having the highest value of matching degree from among second country ETC medications having the certain value of matching degree or higher (operation S530). When the second country ETC medications having the certain value of matching degree or higher do not exist, the global telemedicine system 200 calculates the matching degrees of each medication items with the OTC medications of the OTC medication DB of the second country, based on the analyzed medication detail information, and generate the second country prescription corresponding to the first country language prescription by using, as the second country alternative medication, the second country OTC medication having the highest value of matching degree from among the second country OTC medications having the certain value of matching degree or higher (operation S540). The global telemedicine system 200 provides the detail information of medication items of the second country prescription to the first-country telemedicine service providing hospital 250 after the second country prescription is generated such that the suitability of the second country prescription is verified and the first-country telemedicine service providing hospital 250 manually revises the second country prescription to confirm the second country prescription when necessary (operation S550). Then, the global telemedicine system 200 generates the second country language prescription by converting or automatically translating the language of the confirmed second country prescription from the first country language to the second country language (operation S560). The generated second country language prescription is transmitted to the second-country patient 260 and/or the second-country cooperative hospital 290 (operation S570).

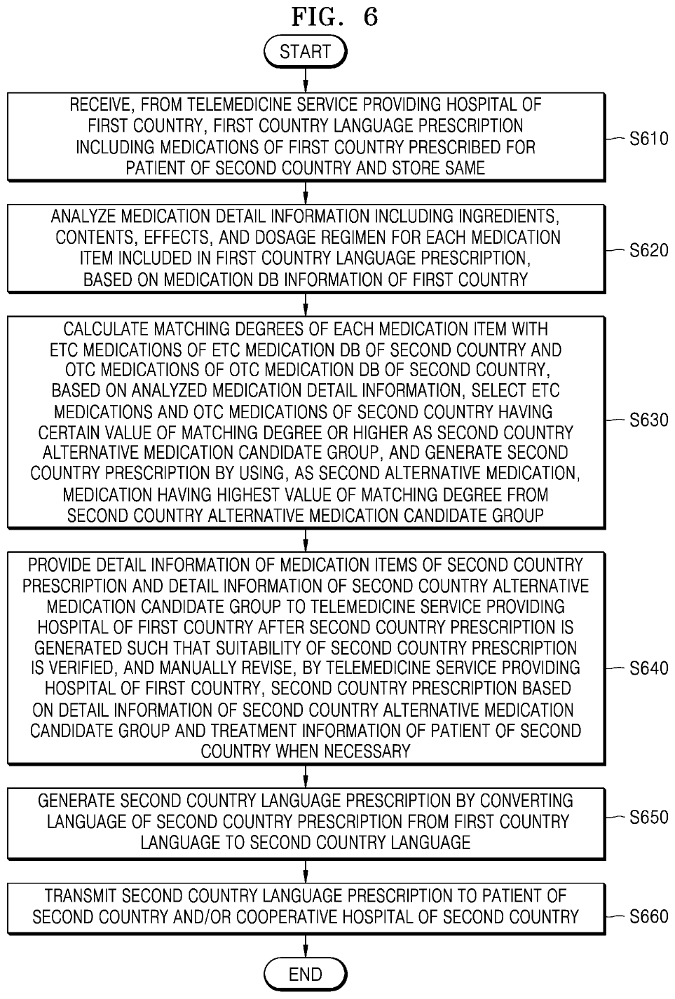

FIG. 6 is a flowchart of a method of performing a country-customized prescription providing service to a patient in a global telemedicine service, according to another embodiment of the present disclosure. The method may be performed by the medication analyzing and matching system 100 of FIGS. 1A and 1B or the global telemedicine system 200 of FIG. 2, according to an embodiment of the present disclosure.

Referring to FIG. 6, in the method according to an embodiment of the present disclosure, the global telemedicine system 200 receives, from the first-country telemedicine service providing hospital 250, the first country language prescription including the medications of the first country prescribed for the second-country patient 260, and stores the same (operation S610). The global telemedicine system 200 analyzes the medication detail information including ingredients, contents, effects, dosage regimen, and additives for each medication item included in the first country language prescription, based on the medication DB information of the first country stored in a connected external institute or an internal DB (operation S620). Then, the global telemedicine system 200 calculates the matching degrees of each medication item with ETC medications of the second-country ETC medication DB 270 and the matching degrees of each medication item with the second-country OTC medications of the OTC medication DB 280, based on the analyzed medication detail information, selects the ETC medications and the OTC medications of the second country having the certain value of matching degree or higher as the second country alternative medication candidate group, and generates the second country prescription by using, as the second country alternative medication, the medication having the highest value of matching degree in the second country alternative medication candidate group (operation S630). After the second country prescription is generated, the global telemedicine system 200 provides the detail information of the medication items of the second country prescription and the detail information of the second country alternative medication candidate group to the first-country telemedicine service providing hospital 250 such that suitability of the second country prescription is verified, and the first-country telemedicine service providing hospital 250 manually revises the second country prescription based on the detail information of the second country alternative medication candidate group, the treatment information of the second-country patient 260, and/or past medical record to confirm the second country prescription when necessary (operation S640). Then, the global telemedicine system 200 generates the second country language prescription by converting or automatically translating the language of the confirmed second country prescription from the first country language to the second country language (operation S650). The global telemedicine system 200 transmits the second country language prescription to the second-country patient 260 of the second country and/or the second-country cooperative hospital 290 (operation S660).

The above description of the present disclosure is provided for illustration, and it will be understood by one of ordinary skill in the art that various changes in form and details may be readily made therein without departing from essential features and the scope of the present disclosure as defined by the following claims. Accordingly, the embodiments described above are examples in all aspects and are not limited. For example, each component described as a single type may be implemented in a distributed manner, and similarly, components described as distributed may be implemented in a combined form.

The scope of the present disclosure is defined by the appended claims, and all changes or modifications within the scope of the appended claims and their equivalents will be construed as being included in the scope of the present disclosure.

According to an embodiment of the present disclosure, a prescription with alternative medications purchasable in a residing country of a patient receiving a telemedicine service, based on a prescription issued by a hospital or doctor of a country providing the telemedicine service, is provided to the patient such that the patient receives stable subsequent treatment by easily obtaining alternative medication in the residing country after receiving the telemedicine service.

The present disclosure enables a patient to easily obtain alternative medications in his/her own country by automatically translating a prescription prepared in a language of a country providing a telemedicine service to a language available in a residing country of the patient receiving the telemedicine service and providing the prescription such that not only the patient but also a medical staff or pharmacist of the residing county of the patient understands the prescription.

The present disclosure enables a patient to easily obtain, from a residing country, medications that best correspond to ethical medications of a prescription issued by a doctor in a country providing a telemedicine service, even when medication items classified as ethical medications that require a prescription and over-the-counter medications purchasable without a prescription are different between the country of a hospital or doctor providing the telemedicine service and the residing county of the patient receiving the telemedicine service.

The present disclosure enables a hospital or doctor that issued an original prescription to check and revise detail information including ingredients, contents, effects, dosage regimen, and additives of alternative medications of a corresponding prescription including medications of a residing country of a patient, the corresponding prescription generated to match the original prescription issued by the hospital or doctor providing a telemedicine service, thereby reducing side effects of medication on the patient, which may occur when the medications of the residing country of the patient are used as alternative medications.

The effects of the present disclosure are not limited thereto and should be understood as including all effects that may be inferred from the configuration of the present disclosure in the detailed description or claims.

What is claimed is:

1. A global medication analyzing and matching system comprising:
a receiving unit configured to receive, from a telemedicine service providing hospital of a first country, a first country language prescription including medications of the first country prescribed for a patient of a second country;
a medication analyzing unit configured to analyze medication detail information including ingredients, contents, effects, dosage regimen, and additives for each of medication items in the first country language prescription, based on received medication database information of the first country;
a medication matching unit configured to search a medication database of the second country for each of the medication items based on the analyzed medication detail information to calculate matching degrees of each of the medication items with found medications in the second country, and generate a second country prescription by using, as a second country alternative medication, a medication of the second country having a highest value of matching degree for each of the medication items;
a language converting unit configured to generate a second country language prescription by converting a language of the second country prescription from a first country language to a second country language;
a transmitting unit configured to transmit the second country language prescription to the patient in the second country; and
a database storing data including the first country language prescription, the second country prescription, and the second country language prescription.

2. The global medication analyzing and matching system of claim 1, wherein
the matching degrees are calculated by calculating similarity between each of the medication items of the first country language prescription and the medications of the second country with respect to the medication detail information including the ingredients, contents, effects, dosage regimen, and additives, according to a matching algorithm of the medication matching unit, wherein a pre-determined individual weight is assigned to each item in the medication detail information.

3. The global medication analyzing and matching system of claim 1, wherein
the medication matching unit is further configured to:
calculate matching degrees of each of the medication items with ethical medications of an ethical medication database of the second country, based on the analyzed medication detail information, and use, as the second country alternative medication, a second country ethical medication having a highest value of matching degree from among second country ethical medications having a certain value of matching degree or higher; and
when the second country ethical medications having the certain value of matching degree or higher do not exist, calculate matching degrees of each of the medication items with over-the-counter medications of an over-the-counter medication database of the second country, based on the analyzed medication detail information, and use, as the second country alternative medication, a second country over-the-counter medication having a highest value of matching degree from among second country over-the-counter medications having the certain value of matching degree or higher.

4. The global medication analyzing and matching system of claim 1, wherein
the medication matching unit is further configured to
provide detail information of medication items of the second country prescription to the telemedicine service providing hospital of the first country after the second country prescription is generated such that suitability of the second country prescription is verified and the telemedicine service providing hospital of the first country manually revises the second country prescription when necessary.

5. The global medication analyzing and matching system of claim 1, wherein
the medication matching unit is further configured to:
calculate matching degrees of each of the medication items with ethical medications of an ethical medication database of the second country and matching degrees of each of the medication items with over-the-counter medications of an over-the-counter medication database of the second country, based on the analyzed medication detail information, select ethical medications and over-the-counter medications of the second country having a certain value of matching degree or higher as second country alternative medication candidate group, and generate the second country prescription by using, as the second country alternative medication, a medication having a highest value of matching degree in the second country alternative medication candidate group, and
provide detail information of medication items of the second country prescription and detail information of the second country alternative medication candidate group to the telemedicine service providing hospital of the first country after the second country prescription is generated such that suitability of the second country prescription is verified and the telemedicine service providing hospital of the first country manually revises the second country prescription based on the detail information of the second country alternative medication candidate group and treatment information of the patient of the second country when necessary.

6. The global medication analyzing and matching system of claim 5, wherein
the medication matching unit is further configured to, when selecting the second country alternative medication candidate group, exclude an ethical medication or over-the-counter medication of the second country containing a drug-sensitive ingredient for the patient of the second country.

7. The global medication analyzing and matching system of claim 5, wherein
the database additionally stores data including the second country alternative medication candidate group and the second country prescription revised by the telemedicine service providing hospital of the first country, and
the global medication analyzing and matching system further comprises a matching model updating unit configured to update a matching algorithm for calculating the matching degrees by learning the data stored in the database.

8. The global medication analyzing and matching system of claim 1, wherein
the transmitting unit is further configured to transmit the second country language prescription to a cooperative hospital of the second country.

9. A global telemedicine service system for providing a telemedicine service between a telemedicine service providing hospital of a first country and a patient of a second country, the global telemedicine service system comprising:
a telemedicine communication unit configured to transmit and receive data such that the telemedicine service is providable between the telemedicine service providing hospital of the first country and the patient of the second country;
a prescription analyzing/matching unit configured to analyze, based on medication database information of the first country, medication detail information including ingredients, contents, effects, dosage regimen, and additives of each of medication items of a first country language prescription including medications received from the telemedicine service providing hospital via the telemedicine communication unit and issued for the patient, calculate matching degrees of each of the medication items with medications of a medication database of the second country, based on the analyzed medication detail information, and generate a second country prescription by using, as a second country alternative medication, a medication of the second country having a highest value of matching degree for each of the medication items;
a language converting unit configured to convert data in a first country language to a second country language and provide the same to the patient, and generate a second country language prescription by converting a language of the second country prescription from the first country language to the second country language, while the telemedicine service is provided via the global telemedicine system; and
a database storing data including data generated when the telemedicine service is provided, information related to the first country language prescription, information related to the second country prescription, and information related to the second country language prescription,
wherein the telemedicine communication unit is further configured to transmit the second country language prescription to the patient of the second country.

10. The global telemedicine system of claim 9, wherein
the matching degrees are calculated by calculating, by the prescription analyzing/matching unit, similarity of each item of the medication detail information including ingredients, contents, effects, dosage regimen, and additives, wherein a pre-determined individual weight is assigned to each item of the medication detail information.

11. The global telemedicine system of claim 9, wherein
the prescription analyzing/matching unit comprises:
a medication analyzing module configured to analyze the medication detail information including ingredients, contents, effects, dosage regimen, and additives for each of medication items in the first country language prescription, based on the medication database information of the first country; and
a medication matching module configured to calculate the matching degrees of each of the medication items with the medications of the medication database of the second country, based on the analyzed medication detail information, and generate the second country prescription by using, as the second country alternative medication, the medication of the second country having the highest value of matching degree for each of the medication items.

12. The global telemedicine system of claim 11, wherein
the medication matching module is further configured to:
calculate matching degrees of each of the medication items with ethical medications of an ethical medication database of the second country, based on the analyzed medication detail information, and use, as the second country alternative medication, a second country ethical medication having a highest value of matching degree from among second country ethical medications having a certain value of matching degree or higher; and
when the second country ethical medications having the certain value of matching degree or higher do not exist, calculate matching degrees of each of the medication items with over-the-counter medications of an over-the-counter medication database of the second country, based on the analyzed medication detail information, and use, as the second country alternative medication, a second country over-the-counter medication having a highest value of matching degree from among second country over-the-counter medications having the certain value of matching degree or higher.

13. The global telemedicine system of claim 11, wherein
the medication matching module is further configured to
provide detail information of medication items of the second country prescription to the telemedicine service providing hospital of the first country after the second country prescription is generated such that suitability of the second country prescription is verified and the telemedicine service providing hospital of the first country manually revises the second country prescription when necessary.

14. The global telemedicine system of claim 11, wherein
the medication matching module is further configured to:
calculate matching degrees of each of the medication items with ethical medications of an ethical medication database of the second country and matching degrees of each of the medication items with over-the-counter medications of an over-the-counter medication database of the second country, based on the analyzed medication detail information, select ethical medications and over-the-counter medications of the second country having a certain value of matching degree or higher as second country alternative medication candidate group, and generate the second country prescription by using, as the second country alternative medication, a medication having a highest value of matching degree in the second country alternative medication candidate group; and
provide detail information of medication items of the second country prescription and detail information of the second country alternative medication candidate group to the telemedicine service providing hospital of the first country after the second country prescription is generated such that suitability of the second country prescription is verified and the telemedicine service providing hospital of the first country manually revises the second country prescription based on the detail information of the second country alternative medication candidate group and treatment information of the patient of the second country when necessary.

15. The global telemedicine system of claim 14, wherein
the medication matching module is further configured to, when selecting the second country alternative medication candidate group, exclude an ethical medication or over-the-counter medication of the second country containing a drug-sensitive ingredient for the patient of the second country.

16. The global telemedicine system of claim 14, wherein
the database additionally stores data including the second country alternative medication candidate group and the second country prescription revised by the telemedicine service providing hospital of the first country, and
the prescription analyzing/matching unit further comprises a matching algorithm updating module configured to update a matching algorithm for calculating the matching degrees by learning the data stored in the database.

17. The global telemedicine system of claim 9, wherein
the telemedicine communication unit further transmits the second country language prescription to a cooperative hospital of the second country.
